(19) United States
(12) Patent Application Publication
(10) Pub. No.: US 2011/0087191 A1
(43) Pub. Date: Apr. 14, 2011

(54) BALLOON CATHETER WITH SHAPE MEMORY SHEATH FOR DELIVERY OF THERAPEUTIC AGENT

(75) Inventor: Torsten SCHEUERMANN, Munich (DE)

(73) Assignee: BOSTON SCIENTIFIC SCIMED, INC., Maple Grove, MN (US)

(21) Appl. No.: 12/902,454

(22) Filed: Oct. 12, 2010

Related U.S. Application Data
(60) Provisional application No. 61/251,422, filed on Oct. 14, 2009.

(51) Int. Cl. A61L 29/16; A61M 25/10

(52) U.S. Cl. 604/509; 604/103.02; 604/103.05

(57) ABSTRACT

A medical device is provided comprising a catheter, a balloon mounted on the catheter, and a sheath comprising a shape memory material, the sheath being located around the balloon. The sheath has a protective condition in which the sheath forms a plurality of pockets in a generally closed position and an activated condition in which the pockets are in a generally open position. A therapeutic agent is located within the generally closed pockets of the sheath when the sheath is in the protective condition. The therapeutic agent is exposed for delivery to a target site when the sheath is transitioned from the protective condition to the activated condition.

### CROSS REFERENCE TO RELATED APPLICATION

The present application claims priority to U.S. provisional application Ser. No. 61/251,422 filed Oct. 14, 2009, the disclosure of which is incorporated herein by reference in its entirety.

### TECHNICAL FIELD

Embodiments of the present disclosure relate to medical devices, more particularly to catheter devices for delivery of therapeutic agent.

### BACKGROUND

Catheters are used in a wide variety of minimally-invasive or percutaneous medical procedures. Balloon catheters having drug coatings may be used to treat diseased portions of blood vessels. Typically, the balloon is inserted through a peripheral blood vessel and then guided via a catheter through the vascular system to the target intravascular site. However, as the balloon travels through the vascular system, the flow of blood may wash away some of the drug coating. In addition, control of the timing, location, and/or duration of the drug release can be an issue.

In some proposed balloon catheters for drug delivery, the coating and/or drug is adhered to the balloon in such a way that upon balloon deflation, the retracting balloon pulls the coating and/or drug back away from the vessel wall. This not only reduces the therapeutic benefit, but it also increases the risk that coating and/or drug can be washed into the blood stream, creating potential complications.

Therefore, to address one or more of the above limitations, there is a need for improved catheter-based devices for drug delivery to an intravascular site.

### SUMMARY

In accordance with certain embodiments of the present disclosure, a medical device is provided comprising a catheter, a balloon mounted on the catheter, and a sheath comprising a shape memory material, the sheath being located around the balloon. The sheath has a protective condition in which the sheath forms a plurality of pockets in a generally closed position and an activated condition in which the pockets are in a generally open position. A therapeutic agent is located within the generally closed pockets of the sheath when the sheath is in the protective condition. The therapeutic agent is exposed for delivery to a target site when the sheath is transitioned from the protective condition to the activated condition.

In accordance with other embodiments of the present disclosure, the sheath in the protective condition may form a plurality of folds, the plurality of pockets being located beneath the plurality of folds. The sheath may be transitioned from the protective condition to the activated condition by any suitable means, such as by heating the sheath or by exposing the sheath to light. The sheath may be transitioned from the protective condition to the activated condition upon or after inflation of the balloon from an unexpanded condition to an expanded condition. The sheath may be attached to the balloon at intervals between the folds around the perimeter of the balloon. The therapeutic agent may be combined with a matrix material to form drug/matrix particles that are located within the generally closed pockets of the sheath when the sheath is in the protective condition. The matrix material in the particles may be biodegradable.

In accordance with other embodiments of the present disclosure, a method of delivering therapeutic agent to a target site is provided. The method comprises providing a medical device comprising a catheter, a balloon mounted on the catheter, and a sheath comprising a shape memory material, the sheath being located around the balloon. The sheath has a protective condition in which the sheath forms a plurality of pockets in a generally closed position and an activated condition in which the pockets are in a generally open position. The method further comprises delivering the balloon to the target site, with the balloon in an unexpanded condition and the sheath in the protective condition, wherein therapeutic agent is located within the generally closed pockets of the sheath when the sheath is in the protective condition. The method further comprises inflating the balloon from its unexpanded condition to an expanded condition and transitioning the sheath from its protective condition to its activated condition, thereby exposing the therapeutic agent for delivery to the target site.

### BRIEF DESCRIPTION OF THE DRAWINGS

FIG. 1 shows a cross-sectional view of a medical device according to an embodiment of the present disclosure, the balloon of the medical device being in an unexpanded condition in a vessel.

FIG. 2 shows the medical device of FIG. 1, with the balloon of the medical device in an inflated, expanded condition.

FIG. 3 shows the medical device of FIG. 1, with the balloon of the medical device in an expanded condition and the sheath of the medical device transitioned to an activated condition exposing the therapeutic agent.

FIG. 4 shows the vessel wall with the therapeutic agent after completion of the procedure illustrated in FIGS. 1-3.

### DETAILED DESCRIPTION

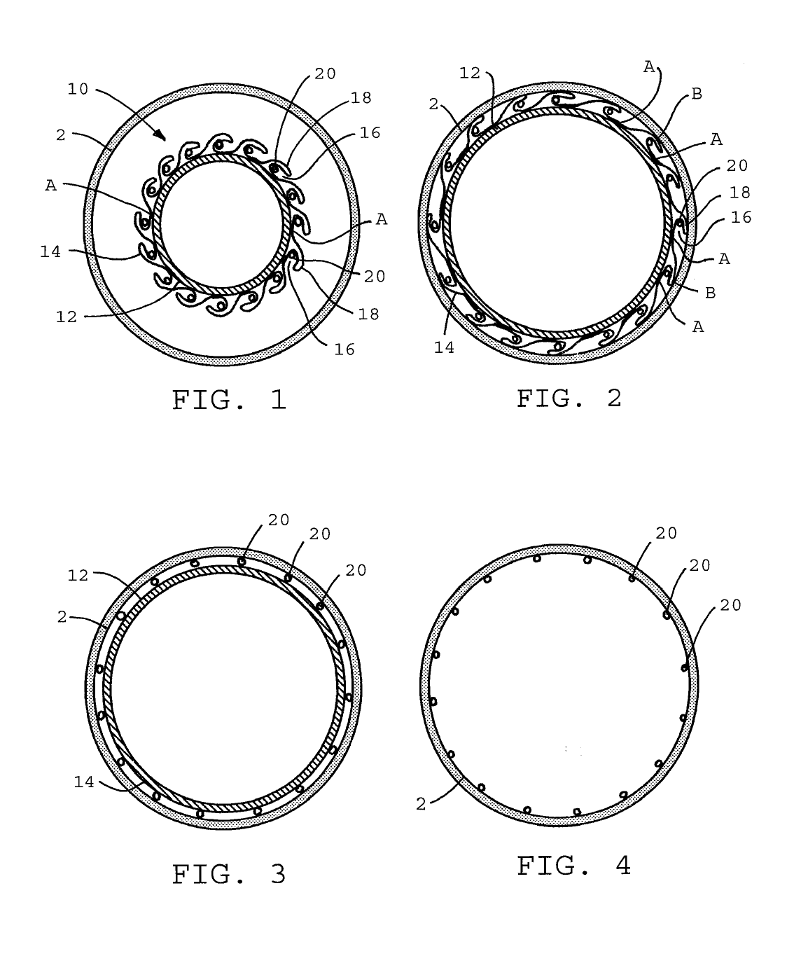

In certain embodiments, such as that illustrated in FIGS. 1-3, catheter devices of the present disclosure use an expandable balloon for delivering a therapeutic agent to a target site in the body. The balloon is designed to be insertable in the body via a catheter. The therapeutic agent can be associated with the balloon in any of various ways, as further described herein. Various mechanisms conventionally used for the delivery, actuation, or expansion (e.g., by inflation) of balloon catheter devices may be used in embodiments of the present disclosure. The balloon catheter may be designed similar to those that have been known in the art, including, but not limited to, angioplasty catheters, stent delivery catheters, inflation catheters, and/or perfusion catheters. The catheter devices of the present disclosure may be used in conjunction with other drug delivery devices, such as stents.

FIG. 1 shows a cross-sectional view of a medical device 10 according to an embodiment of the present disclosure, positioned inside a vessel 2. The medical device 10 comprises a catheter as is known in the art, a balloon 12 mounted on the catheter, and a sheath 14 comprising a shape memory material, the sheath 14 being located around the balloon 12. In FIG. 1, the balloon 12 of the medical device 10 is in a deflated, unexpanded condition inside the vessel 2.

In FIG. 1, the sheath 14 is in a protective condition in which the sheath 14 forms a plurality of pockets 16 in a generally closed position. A therapeutic agent is located within the generally closed pockets 16 of the sheath 14 when the sheath 14 is in the protective condition.

The therapeutic agent or drug that is used can be selected depending on the treatment desired. For example, drugs useful as anti-proliferative agents or anti-restenosis agents may be desired. Specific examples of drugs that may be used include paclitaxel, sirolimus and everolimus, as well as further drugs identified herein.

In the embodiment of FIG. 1, the therapeutic agent is in the form of, or is carried as part of, particles 20 that are located within the generally closed pockets 16 of the sheath 14 when the sheath 14 is in the protective condition. The particles 20 can comprise just a drug alone, e.g., in crystal form, a drug mixture, or a drug that is mixed with or otherwise carried by a matrix material to form particles 20. If a matrix material is used, the matrix material in the particles 20 may be biodegradable. For example, the matrix material may be a biodegradable polymer such as PLLA or PLGA. Alternatively, the matrix material may comprise albumin, such as in Abraxane® (Astra-Zeneca) (albumin-bound paclitaxel). Further information regarding Abraxane® can be found, for example, at http://www.rxlist.com/cgi/generic/abraxane.htm.

The sheath 14 is made of a shape memory material. In a first condition, such as that shown in FIG. 1, the sheath 14 forms a plurality of pockets 16 in a generally closed position. In the embodiment of FIG. 1, the sheath 14 in this condition forms a plurality of folds 18, the plurality of pockets 16 being located beneath the plurality of folds 18. It will be appreciated that in FIG. 1 as well as in the other figures the illustration is drawn for clarity, and the dimensions of the actual device may not be as shown. In addition, the folds 18 may be completely folded over to contact the balloon and completely enclose the particles 20.

The sheath 14 may be attached to the balloon 12 at intervals between the folds 18 around the perimeter of the balloon 12. For example, the sheath 14 may be bonded to the balloon 12 at the attachment areas labeled A in FIG. 1. Only two attachment areas A are labeled, but attachment areas may be located between each pair of adjacent folds 18. Alternatively, fewer attachment areas may be used, such that attachment areas are located only between some adjacent folds 18.

The shape memory material of the sheath 14 may be a shape memory polymer as is known in the art. Shape memory polymers that undergo a change in shape when acted upon by an external stimulus are available. The shape memory materials can be manufactured and shaped in such a way that they are “programmed” to take on a particular shape under certain conditions. For example, shape memory polymers are available that undergo a thermally-induced shape change when heated above a transition temperature. In addition, shape memory polymers are available that undergo a light-induced shape change when acted upon by a light stimulus. Suitable shape memory polymers that may be used to form sheath 14 in FIG. 1 include shape memory polymers such as those described in U.S. Patent Application No. 2008/0312733 and U.S. Patent Application No. 2003/0055198, the disclosures of which are hereby incorporated by reference herein. Some information regarding shape memory polymers as known in the art is available in Lendlein, “Shape-Memory Polymers,” Angewandte Chemie International Edition, vol. 41, pp. 2034-2057 (2002), and in Lendlein, “Light-Induced Shape-Memory Polymers,” Nature, vol. 434, pp. 879-882 (2005). The company Mnemoscience GmbH in Aachen, Germany, is a company that can provide shape memory polymers (see, generally, http://www.mnemoscience.de).

The shape memory material of the sheath 14 in FIG. 1 can be programmed to undergo a shape change from the protective condition shown in FIG. 1 to an activated condition in which the pockets 16 open and the therapeutic agent is exposed for delivery to a target site. The sheath 14 may be transitioned from the protective condition to the activated condition upon or after inflation of the balloon 12 from its unexpanded condition as shown in FIG. 1 to an expanded condition.

FIG. 2 shows the medical device 10 of FIG. 1 with the balloon 12 of the medical device 10 in an inflated, expanded condition. As discussed herein, various mechanisms conventionally used for inflation of balloon catheter devices may be used to inflate the balloon 12.

In FIG. 2, the sheath 14 has stretched to accommodate the enlarged balloon diameter. The sheath 14 may undergo transition to expose the pockets 16 during balloon inflation and/or after balloon inflation. In the embodiment shown in FIG. 2, the pockets 16 are still generally covered by the folds 18 such that the particles 20 have not yet been exposed for delivery to the vessel 2.

In the embodiment shown in FIG. 2, the outside of the folds 18 contact the inner wall of the vessel 2 at contact areas, labeled B in FIG. 2. Only two contact areas B are labeled in FIG. 2, but it can be seen that each of the folds 18 in FIG. 2 contacts the vessel wall at a contact area B. As illustrated in FIG. 2, the distance along the surface of the sheath 14 from a contact area B to an adjacent attachment area A, where the sheath 14 is attached to the balloon 12, is shorter in one direction than in the opposite direction. That is, the amount of sheath material from a contact area B in a counter-clockwise direction (in FIG. 2) to the adjacent attachment area A is less than the amount of sheath material from that contact area B in a clockwise direction (in FIG. 2) to the adjacent attachment area A. This is due to the presence of the folds 18.

FIG. 3 shows the medical device 10 after the sheath 14 has transitioned to its activated condition to expose the therapeutic agent. The sheath 14 may be transitioned, for example, by a heat stimulus. The sheath material may be a shape memory polymer that shrinks due to an increase in temperature, such as an increase to body temperature or a higher temperature. To control the shape memory polymer transition, the inflation fluid in the balloon catheter can be slightly higher than body temperature. When the sheath 14 is heated above its transition temperature, the shape memory material can shrink, causing the folds 18 to get smaller and, in the illustrated embodiment, practically disappear by virtue of the material shrinking tight around the balloon 12. As this happens, the pockets 16 open up, thereby exposing the particles 20. FIG. 3 illustrates the sheath 14 in an activated condition in which the pockets are in a fully open position, but in the activated condition the pockets need only generally be opened sufficiently to allow delivery of the particles 20 comprising the therapeutic agent.

The pressure of the balloon 12 and the shrinkage of the sheath 14 can cause the particles 20 to be exposed and pressed into the vessel wall. An increase in the pressure in the balloon can increase a force pressing the particles 20 into the vessel wall.

In addition, in certain embodiments such as the illustrated embodiment, the balloon may undergo a slight rotation due to the shrinkage of the sheath 14. For example, as discussed herein, at the stage in the process illustrated in FIG. 2, the amount of sheath material from a contact area B in a counter-clockwise direction (in FIG. 2) to the adjacent attachment area A is less than the amount of sheath material from that contact area B in a clockwise direction (in FIG. 2) to the adjacent attachment area A. As the shape memory material of the sheath 14 shrinks, the balloon may be rotated slightly clockwise. This is because the material at the contact area B will generally be held against rotation by the pressing of the sheath 14 against the vessel wall and friction, and the uneven distribution of material between the contact area and adjacent attachment areas A will create uneven forces on the balloon. In the illustrated example, the contact area A in a counter-clockwise direction from a contact area B will be pulled toward the contact area B due to the shrinkage of the sheath material. The contact area A in a clockwise direction from a contact area B will not be similarly pulled because the shrinkage of the material can be compensated at least in part by the take-up of the slack in the material caused by the fold 18. The amount of the rotation can vary, but it may be, for example, on the order of a few degrees or less, such as 1 to 2 degrees.

In the case of an embodiment in which the balloon rotates, the rotation of the balloon can assist in forcing the particles into the vessel wall. The balloon rotation and the balloon pressure can smear or force the particles into the calcified plaque of the vessel wall at the stenosis area.

FIG. 4 shows the vessel wall 2 with the particles 20 comprising the therapeutic agent after completion of the procedure illustrated in FIGS. 1-3. The balloon 12 has been deflated, and the catheter has been withdrawn from the vessel, leaving behind the particles 20 comprising the therapeutic agent.

In embodiments in which the particles 20 comprise a biodegradable matrix material, after delivery the biodegradable material can erode and the therapeutic agent can be released. The release mechanism can be diffusion of the drug through the matrix material and/or erosion of the matrix material which releases the drug.

The matrix material used with the therapeutic agent may be chosen for its drug release characteristics. For example, certain polymers will facilitate a burst release and others will facilitate a more sustained release. In some embodiments, the particles under the folds of a sheath as shown in FIG. 1 may be different such that, for example, some of the particles may be burst release particles while others are sustained release particles. The composition and distribution of the particles can be adjusted depending on the desired treatment and result.

In addition to having the sheath transitioned from the protective condition to the activated condition by thermal activation, the sheath may be transitioned from the protective condition to the activated condition by any other suitable means, such as by exposing the sheath to light. The balloon may contain a light source with a connection through the shaft to a power supply. When the light source emits appropriate radiation, the shape memory material shrinks as previously programmed.

The method of using a medical device 10 as illustrated in FIGS. 1-3 will be understood by persons of ordinary skill in the art. A physician can deliver the balloon 12 to the desired target site by means known in the art for delivering balloon catheters. The balloon 12 is delivered to the target site with the balloon 12 in an unexpanded condition and the sheath 14 in the protective condition, with the particles 20 located within the generally closed pockets 16 of the sheath 14, as shown in FIG. 1. Once at the desired site, the balloon 12 is inflated from its unexpanded condition to an expanded condition, and the sheath 14 is transitioned as described herein (e.g., by applying heat and/or light) from its protective condition to its activated condition, thereby exposing the particles 20 for delivery to the target site. Once the inflation and delivery steps are completed, the balloon 12 is deflated and the catheter is withdrawn.

Devices and methods in accordance with embodiments of the present disclosure can have one or more advantages. For example, the particles comprising therapeutic agent need not be adhered to the balloon but may simply be placed under the folds. Alternatively, they may be loosely adhered to the balloon. In either case, the particles can be held within the folds such that, after balloon inflation and delivery of the particles to the vessel wall, the particles have no significant issue of sticking to the balloon when the balloon is deflated. In some prior proposed balloon catheters for drug delivery, the coating and/or drug is adhered to the balloon in such a way that upon balloon deflation, the retracting balloon can pull the coating and/or drug back away from the vessel wall. This not only reduces the therapeutic benefit, but it also increases the risk that coating and/or drug can be washed into the blood stream, creating potential complications. In certain embodiments of the present disclosure, because the particles comprising the therapeutic agent can be generally held in place by the pockets of the shape memory sheath, the particles comprising the therapeutic agent may be unadhered or only loosely adhered to the balloon, thereby substantially avoiding these issues.

In addition, in certain embodiments of the present disclosure, the pockets or folds of the sheath can protect the particles comprising the therapeutic agent during tracking of the balloon to the target site. Because the pockets/folds can protect the particles during tracking to the target site, the pockets/folds can help substantially avoid the issue of drug or a drug/matrix material becoming dislodged as the balloon travels through the vascular system, which could allow the flow of blood to wash away some of the drug and/or matrix material.

The folds/pockets that can be controlled by activation of the shape memory sheath also allow the user to control the timing and location of the drug release. For example, when the sheath is activated by heat and/or light, the heat and/or light can be applied only at the desired time of drug delivery, and only when the balloon is at the target site. In addition, the user has control over the duration of the application of the heat and/or light, and the heat and/or light can be applied only for the desired duration.

As described herein, the sheath shape memory material can be “programmed” to have the shape of the protective shape (with a plurality of pockets in a generally closed position) when the sheath is below the transition temperature and the activated shape (with the pockets in a generally open position) when the sheath is above the transition temperature. The protective shape can be the shape the sheath material takes at room temperature.

In one example of manufacturing a medical device such as the medical device 10 of FIGS. 1-3, a conventional balloon is inflated (e.g., with gas). Then, the environment and balloon is heated (e.g., to 40 degrees Celsius) by suitable means such as IR radiation or hot air. A shape memory sheath made of a shape memory polymer is provided with “programmed” folds as described herein. The shape memory sheath with the “programmed” folds is slid or placed over the balloon, and it shrinks to the activated open shape due to the elevated temperature (which is over the transition temperature which may be, e.g., 38 degrees Celsius). At the areas of the sheath that will be generally covered by the folds (when the temperature is below the transition point), the particles comprising the therapeutic agent are deposited. The particles can comprise, e.g., paclitaxel, sirolimus (rapamycin), tacrolimus, everolimus, biolimus and/or zotarolimus and a bioabsorbable matrix such as PLGA or PLA. The depositing of the particles may be by any suitable means, such as by rolling or stamping or through a “ProtoPrint” process such as described at http://www.vdivde-it.de/innonet/projekte/in_pp151_protoprint.pdf.

The sheath may be bonded to the balloon at desired intervals (such as at attachment areas A described herein) by a laser welding process. These areas can be where the sheath contacts the balloon between the folds (when the temperature is below the transition point). Then, the assembly can be cooled below the transition point, by which the shape memory material changes to the protective shape, and the folds cover the particles comprising the therapeutic agent. The balloon can then be deflated and folded in a conventional way.

It will be appreciated that other embodiments can be created with variations. Some example variations include, but are not limited to, changing the size, shape, and/or number of folds. The folds may extend the entire length of the balloon or only along part of the length of the balloon. The folds may extend in an linear direction parallel to the axis of the balloon or in another manner, such as in a helical direction around the balloon.

The therapeutic agent used in embodiments of the present disclosure may be any pharmaceutically-acceptable agent suitable for the intended application, such as a drug, a non-genetic therapeutic agent, a biomolecule, a small molecule, or cells. Example drugs include anti-proliferative agents or anti-restenosis agents such as paclitaxel, sirolimus (rapamycin), tacrolimus, everolimus, biolimus and zotarolimus.

Exemplary non-genetic therapeutic agents include anti-thrombogenic agents such heparin, heparin derivatives, prostaglandin (including micellar prostaglandin E1), urokinase, and PPack (dextrophenylalanine proline arginine chloromethylketone); anti-proliferative agents such as enoxaparin, angiopeptin, sirolimus (rapamycin), tacrolimus, everolimus, zotarolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis (2-aminoethyl) ethyleneglycol-N,N,N′,N′-tetraacetic acid and mixtures thereof; antibiotics such as gentamycin, rifampin, minocyclin, and ciprofloxacin; antibodies including chimeric antibodies and antibody fragments; anesthetic agents such as lidocaine, bupivacaine, and ropivacaine; nitric oxide; nitric oxide (NO) donors such as linsidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, warfarin sodium, Dicumarol, aspirin, prostaglandin inhibitors, platelet aggregation inhibitors such as cilostazol and tick antiplatelet factors; vascular cell growth promotors such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; inhibitors of heat shock proteins such as geldanamycin; angiotensin converting enzyme (ACE) inhibitors; beta-blockers; βAR kinase (βARK) inhibitors; phospholamban inhibitors; protein-bound particle drugs such as ABRAXANE™; structural protein (e.g., collagen) cross-link breakers such as alagebrium (ALT-711); and any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include serca-2 protein, monocyte chemoattractant proteins (MCP-1) and bone morphogenic proteins (“BMPs”), such as, for example, BMP-2, BMP-3, BMP-4, BMP-5, BMP-6 (VGR-1), BMP-7 (OP-1), BMP-8, BMP-9, BMP-10, BMP-11, BMP-12, BMP-13, BMP-14, BMP-15. Preferred BMPs are any of BMP-2, BMP-3, BMP-4, BMP-5, BMP-6, and BMP-7. These BMPs can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them. Non-limiting examples of genes include survival genes that protect against cell death, such as anti-apoptotic Bcl-2 family factors and Akt kinase; serca 2 gene; and combinations thereof. Non-limiting examples of angiogenic factors include acidic and basic fibroblast growth factors, vascular endothelial growth factor, epidermal growth factor, transforming growth factors α and β, platelet-derived endothelial growth factor, platelet-derived growth factor, tumor necrosis factor α, hepatocyte growth factor, and insulin-like growth factor. A non-limiting example of a cell cycle inhibitor is a cathespin D (CD) inhibitor. Non-limiting examples of anti-restenosis agents include p15, p16, p18, p19, p21, p2′7, p53, p57, Rb, nFkB and E2F decoys, thymidine kinase and combinations thereof as well as other agents useful for interfering with cell proliferation.

Exemplary small molecules include hormones, nucleotides, amino acids, sugars, and lipids and compounds having a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or genetically engineered. Non-limiting examples of cells include side population (SP) cells, lineage negative (Lin⁻) cells including Lin⁻CD34⁻, Lin⁻CD34⁺, Lin⁻ cKit⁺, mesenchymal stem cells including mesenchymal stem cells with 5-aza, cord blood cells, cardiac or other tissue derived stem cells, whole bone marrow, bone marrow mononuclear cells, endothelial progenitor cells, skeletal myoblasts or satellite cells, muscle-derived cells, go cells, endothelial cells, adult cardiomyocytes, fibroblasts, smooth muscle cells, adult cardiac fibroblasts +5-aza, genetically modified cells, tissue engineered grafts, MyoD scar fibroblasts, pacing cells, embryonic stem cell clones, embryonic stem cells, fetal or neonatal cells, immunologically masked cells, and teratoma-derived cells. Any of the therapeutic agents may be combined to the extent such combination is biologically compatible.

The foregoing description and examples have been set forth merely to illustrate the present disclosure and are not intended to be limiting. Each of the disclosed aspects and embodiments of the present disclosure may be considered individually or in combination with other aspects, embodiments, and variations of the present disclosure. Modifications of the disclosed embodiments may be made within the scope of the present disclosure as defined in the claims.

What is claimed is:

1. A medical device comprising:
a catheter;
a balloon mounted on the catheter;
a sheath comprising a shape memory material, the sheath being located around the balloon; and
a therapeutic agent;
wherein the sheath has a protective condition in which the sheath forms a plurality of pockets in a generally closed position and an activated condition in which the pockets are in a generally open position;
wherein the therapeutic agent is located within the generally closed pockets of the sheath when the sheath is in the protective condition; and
wherein the therapeutic agent is exposed for delivery to a target site when the sheath is transitioned from the protective condition to the activated condition.

2. The medical device of claim 1, wherein the sheath in the protective condition forms a plurality of folds, the plurality of pockets being located beneath the plurality of folds.

3. The medical device of claim 1, wherein the sheath is transitioned from the protective condition to the activated condition by heating the sheath.

4. The medical device of claim 1, wherein the sheath is transitioned from the protective condition to the activated condition by exposing the sheath to light.

5. The medical device of claim 1, wherein the sheath is transitioned from the protective condition to the activated condition upon inflation of the balloon from an unexpanded condition to an expanded condition.

6. The medical device of claim 1, wherein the sheath is transitioned from the protective condition to the activated condition after inflation of the balloon from an unexpanded condition to an expanded condition.

7. The medical device of claim 1, wherein the sheath is attached to the balloon at intervals between the folds around the perimeter of the balloon.

8. The medical device of claim 1, wherein the sheath is not attached to the balloon.

9. The medical device of claim 1, wherein the therapeutic agent is combined with a matrix material to form particles that are located within the generally closed pockets of the sheath when the sheath is in the protective condition.

10. The medical device of claim 9, wherein the matrix material in the particles is biodegradable.

11. A method of delivering therapeutic agent to a target site, the method comprising:
providing a medical device comprising:
a catheter;
a balloon mounted on the catheter;
a sheath comprising a shape memory material, the sheath being located around the balloon; and
a therapeutic agent;
wherein the sheath has a protective condition in which the sheath forms a plurality of pockets in a generally closed position and an activated condition in which the pockets are in a generally open position;
delivering the balloon to the target site, with the balloon in an unexpanded condition and the sheath in the protective condition, wherein the therapeutic agent is located within the generally closed pockets of the sheath when the sheath is in the protective condition;
inflating the balloon from its unexpanded condition to an expanded condition; and
transitioning the sheath from its protective condition to its activated condition, thereby exposing the therapeutic agent for delivery to the target site.

12. The method of claim 11, wherein the sheath in the protective condition forms a plurality of folds, the plurality of pockets being located beneath the plurality of folds.

13. The method of claim 11, wherein the sheath is transitioned from the protective condition to the activated condition by heating the sheath.

14. The method of claim 11, wherein the sheath is transitioned from the protective condition to the activated condition by exposing the sheath to light.

15. The method of claim 11, wherein the therapeutic agent is combined with a matrix material to form particles that are located within the generally closed pockets of the sheath when the sheath is in the protective condition.

16. The method of claim 15, wherein the matrix material in the particles is biodegradable.

17. A method of delivering therapeutic agent to a target site, the method comprising:
providing a medical device comprising:
a catheter;
a balloon mounted on the catheter;
a sheath comprising a shape memory material, the sheath being located around the balloon; and
a therapeutic agent;
wherein the sheath has a protective condition in which the sheath forms a plurality of pockets in a generally closed position and an activated condition in which the pockets are in a generally open position;
delivering the balloon to the target site, with the balloon in an unexpanded condition and the sheath in the protective condition, wherein the therapeutic agent is located within the generally closed pockets of the sheath when the sheath is in the protective condition;
inflating the balloon from its unexpanded condition to an expanded condition;
transitioning the sheath from its protective condition to its activated condition, thereby exposing the therapeutic agent for delivery to the target site; and
rotating the balloon with respect to the target site.

18. The method of claim 17, wherein the step of rotating the balloon with respect to the target site provides a rotational force on the therapeutic agent against tissue at the target site.

19. The method of claim 18, wherein the therapeutic agent is combined with a matrix material to form particles that are located within the generally closed pockets of the sheath when the sheath is in the protective condition.

20. The method of claim 19, wherein the matrix material in the particles is biodegradable.
